(19) United States
(12) Patent Application Publication
(10) Pub. No.: US 2002/0168343 A1
(43) Pub. Date: Nov. 14, 2002

(54) Combined transductional and transcriptional targeting system for improved gene delivery

(76) Inventors: David T. Curiel, Birmingham, AL (US); Paul N. Reynolds, Birmingham, AL (US)

Correspondence Address: Benjamin Aaron Adler, ADLER & ASSOCIATES, 8011 Candle Lane, Houston, TX, 77071, US

(21) Appl. No.: 10/075,322

(22) Filed: Feb. 14, 2002

Related U.S. Application Data
(60) Provisional application No. 60/268,544, filed on Feb. 14, 2001.

(51) Int. Cl. A61K 48/00; C12N 15/861

(52) U.S. Cl. 424/93.2; 514/44; 435/456; 435/320.1

(57) ABSTRACT

The present invention provides a gene delivery system that combines transductional targeting via binding to angiotensin converting enzyme (ACE) expressed on pulmonary endothelial cells with transcriptional targeting using the vascular endothelial growth factor type 1 receptor (flt-1) promoter. Compared to either approach used alone, this combined targeting approach resulted in a dramatic improvement in the target: non-target transgene expression ratio in vivo, thereby improving the prospects for pulmonary vascular gene therapy and establishing a fundamental principal for the use of targeting strategies generally.

### CROSS-REFERENCE TO RELATED APPLICATION

This non-provisional patent application claims benefit of provisional patent application U.S. Serial No. 60/268,544, filed Feb. 14, 2001, now abandoned.

### FEDERAL FUNDING LEGAND

This invention was produced in part using funds obtained through grants from the National Institutes of Health. Consequently, the federal government has certain rights in this invention.

### BACKGROUND OF THE INVENTION

1. Field of the Invention

The present invention relates generally to the field of gene therapy vectorology. More specifically, the present invention relates to a combined transductional and transcriptional targeting approach for gene delivery in vivo by an adenoviral vector.

2. Description of the Related Art

Gene therapy may offer new options for the treatment of pulmonary vascular diseases, conditions for which conventional therapies are limited (1). The recent discovery of the genetic basis for primary pulmonary hypertension, along with a lack of effective conventional therapies for this disease, provide a clear rationale for the development of improved pulmonary endothelial gene transfer technologies. Strategies to efficiently and specifically direct therapeutic transgene expression to the pulmonary vascular endothelium would help to ensure that the full potential of this approach is realized.

Adenoviral vectors are attractive candidates for this task in view of their generally high in vivo gene delivery efficacy compared to other vectors (2, 3). However, conventional adenoviral vectors do not achieve widespread pulmonary endothelial gene delivery following intravascular administration in rodent and primate models (4). The use of these agents is compromised by the natural tropism of the virus for the coxsackie/adenoviral receptor (CAR) (5, 6); many tissues lack accessible coxsackie/adenoviral receptor and are therefore poorly transduced. On the other hand, the liver expresses high levels of the coxsackie/adenoviral receptor, which contributes to its high susceptibility to ectopic transduction and the risk of deleterious consequences (7). In fact, hepatic sequestration of adenoviral vectors is one of the main limitations to the systemic use of these agents for a variety of applications, including pulmonary vascular gene delivery. To overcome these limitations, strategies have been devised to impart specific targeting properties to adenoviral vectors, both to improve efficacy at the target site and reduce ectopic transgene expression. These efforts include both transductional and transcriptional approaches.

Transductional targeting is based upon the alteration of the natural infection pathway of the adenoviral vector (8). This infection normally involves a two-step process, whereby cellular attachment is achieved by binding of the knob domain of the adenoviral fiber to the coxsackie/adenoviral receptor, followed by internalization of the virion via an interaction between cell-surface integrins and an Arg-Gly-Asp (RGD) motif in the adenoviral penton base (9). Thus, to alter tropism, efforts have logically focused on modifying the adenoviral knob domain. This has been achieved through the use of bi-specific adaptors that simultaneously bind to knob, neutralise coxsackie/adenoviral receptor recognition and impart new tropism (10), or by direct genetic modification of the knob domain itself (11).

Recently, an adaptor approach has been described to redirect infection via attachment to angiotensin converting enzyme (ACE), a membrane bound ectoezyme highly expressed on pulmonary endothelial cells (12). This strategy achieved enhanced gene delivery to pulmonary endothelial cells in vivo, while simultaneously reducing transgene expression in the liver, the first demonstration of targeting via the systemic route. However, limitations to this approach were noted. Specifically, the level of liver transgene expression remained high in absolute terms. Genetic modifications of adenoviral to ablate coxsackie/adenoviral receptor recognition (at least in the absence of an alternate targeting ligand) have not reduced hepatic transgene expression. Secondary interactions between an Arg-Gly-Asp (RGD) motif in the adenoviral penton base and cell-surface integrins (which normally mediate internalization of the virion after primary attachment to coxsackie/adenoviral receptor) may account for some of the residual hepatocyte transduction. Other less well-defined mechanisms may also be involved. These findings suggest the need for complementary approaches.

Transcriptional targeting involves the use of cell-specific promoters (13). There have been considerable advances in this area recently, with the identification of several promoters that retain specificity in adenoviral vectors (14, 15). Recently the use of the promoter for the vascular endothelial growth factor type 1 receptor (flt-1 promoter) in an adenoviral vector was described, showing both a high level of activity in endothelial cells and a low level of activity in hepatocytes in culture and the liver in vivo (16). Nevertheless, this approach in isolation is of no benefit for pulmonary endothelial application if the cells are poorly transduced.

Thus, the prior art is deficient in a method for gene delivery in vivo by an adenoviral vector with improved efficacy at the target site and reduced ectopic transgene expression. The present invention fulfills this long-standing need and desire in the art.

### SUMMARY OF THE INVENTION

The current invention demonstrates that through a judicious combination of approaches, a high degree of efficiency and specificity of transgene expression in target cells in vivo was achieved, thereby establishing an important new paradigm in gene delivery technology. Although this new gene delivery paradigm is established in the context of the transduction of pulmonary vascular endothelium, the current application has far-reaching implications for the broader development of gene delivery systems for virtually any in vivo application.

The present invention demonstrates that adenoviral vector targeting to pulmonary endothelium can be substantially improved by a combination of transductional and transcriptional approaches. In fact, the validity of this basic concept has not previously been established for any target cell due to the lack of complementary transductional and transcriptional strategies that have fidelity in vivo. The present invention combines two recently described strategies for the targeting of endothelial cells, namely transductional targeting via binding to angiotensin converting enzyme (ACE) and transcriptional targeting using the vascular endothelial growth factor type 1 receptor (flt-1) promoter. Compared to either approach used alone, this combined targeting approach resulted in a dramatic, synergistic, improvement in the target to non-target transgene expression ratio in vivo, thereby improving the prospects for pulmonary vascular gene therapy and establishing a fundamental principle for the use of targeting strategies generally.

Thus, the present invention is directed to an adenoviral vector that mediates increased gene delivery in vivo. This vector comprises a targeting component that targets the vector to specific target cells and a tissue-specific promoter that drives the expression of a transgene carried by the vector in the target cells. In general, the targeting component can be a targeting ligand incorporated into the fiber or other capsid protein of the adenoviral vector by genetic mutation. Alternatively, the targeting component can be a bi-specific molecule that binds to the knob or other capsid protein of the adenoviral vector and a molecule expressed on the target cells. In one embodiment, when the target cells are pulmonary endothelial cells, the adenoviral vector comprises a vascular endothelial growth factor type 1 receptor promoter and a bi-specific antibody conjugate linking a Fab fragment of an anti-Ad5 knob antibody 1D6.14 with an anti-angiotensin converting enzyme (ACE) antibody 9B9.

The present invention is also directed to an improved method of gene delivery using an adenoviral vector, comprising the step of: contacting target cells with an adenoviral vector comprising a targeting component that targets the vector to specific target cells and a tissue-specific promoter that drives the expression of transgene carried by the vector in the target cells, wherein the adenoviral vector has enhanced targeting specificity to the target cells and results in reduced transgene expression in non-target cells. In general, the targeting component of the adenoviral vector can be a targeting ligand incorporated into the fiber protein or other capsid protein of the adenoviral vector by genetic mutation. Alternatively, the targeting component can be a bi-specific molecule that binds to the knob protein or other capsid protein of the adenoviral vector and a molecule expressed on the target cells. In one embodiment, when the target cells are pulmonary endothelial cells, the adenoviral vector comprises a vascular endothelial growth factor type 1 receptor promoter and a bi-specific antibody conjugate linking a Fab fragment of an anti-Ad5 knob antibody 1D6.14 with an antiangiotensin converting enzyme (ACE) antibody 9B9.

Other and further aspects, features, and advantages of the present invention will be apparent from the following description of the presently preferred embodiments of the invention. These embodiments are given for the purpose of disclosure.

### BRIEF DESCRIPTION OF THE DRAWINGS

So that the matter in which the above-recited features, advantages and objects of the invention, as well as others which will become clear, are attained and can be understood in detail, more particular descriptions of the invention briefly summarized above may be had by reference to certain embodiments thereof which are illustrated in the appended drawings. These drawings form a part of the specification. It is to be noted, however, that the appended drawings illustrate preferred embodiments of the invention and therefore are not to be considered limiting in their scope.

FIG. 1 shows AdfltLuc vs AdCMVLuc transgene expression in murine endothelial cells. The 1P-1B cell line was plated at 50,000 cells per well in 24 well plates, then transduced using various doses of either AdfltLuc or AdCMVLuc (containing the strong but non-specific cytomegalovirus promoter) as indicated. Luciferase assay was performed 24 hours later. These data illustrate the basic functionality of the AdfltLuc vector and indicate the strength of the flt-1 promoter relative to CMV in this line.

FIG. 2 shows AdfltCEA vs AdCMVCEA transgene expression in murine endothelial cells. The 1P-1B cell line was plated at 50,000 cells per well in 24 well plates, then transduced using various doses of either AdfltCEA or AdCMVCEA as indicated. Forty eight hours later the cells were stained using an anti-CEA antibody and DAB detection, positive signal is shown by brown precipitate.

FIG. 2A: Uninfected cells.

FIG. 2B: AdCMVCEA infected cells.

FIG. 2C: AdfltCEA infected cells. These data show the basic functionality and strength of the AdfltCEA vector.

FIG. 3 shows luciferase gene delivery in vivo. Rats were injected (tail vein) with 5×10⁹ pfu of AdCMVLuc or AdfltLuc, either alone (FIG. 3A, FIG. 3C) or in combination with the pulmonary endothelial targeting conjugate Fab-9B9 (FIG. 3B, FIG. 3D), then sacrificed three days later and luciferase activity was determined. Data are means ±SD of 8-10 rats per group. These results clearly show the striking, synergistic improvement in transgene expression in the target organ which is achieved with the combined targeting approach.

FIG. 4 shows targeting fidelity is maintained upon left ventricular injection. Rats were injected via either the tail vein (FIG. 4A) or left ventricle (FIG. 4B) with 1×10¹¹ viral particles of AdfitLuc+Fab-9B9, and luciferase activity was determined three days later. Data are means ±s.d. of four rats per group. FIG. 4C shows left ventricular injection of AdfltLuc alone.

FIG. 5 shows improved selectivity at high vector dose. Rats were injected (tail vein) with 3×10¹¹ viral particles of AdfltLuc, either alone (FIG. 5A) or in combination with the pulmonary endothelial targeting conjugate Fab-9B9 (FIG. 5B), then killed three days later and luciferase activity was determined. Data are means ±s.d. of four rats per group.

FIG. 6 shows the distribution of transgene expression within different organs. Rats were injected via the tail vein with 3×10¹⁰ pfu of either AdCMVCEA+Fab9B9 or AdfltCEA+Fab-9B9, then sacrificed 4 days later. Panels show staining for CEA transgene expression as shown by green fluorescence.

FIG. 6A, FIG. 6C and FIG. 6E are sections of lung, liver and spleen, respectively from a rat that received AdCMVCEA+Fab9B9.

FIG. 6B, FIG. 6D and FIG. 6F are corresponding sections from a rat that received AdfltCEA+Fab-9B9. Nuclei were stained using Hoescht 33342.

FIG. 7 shows transgene expression in lung. High power view of lung sections from a rat that received AdfltCEA+Fab9B9, clearly showing transgene expression (green fluorescence) in the endothelium of alveolar capillaries (FIG. 7A) and small and medium sized vessels (FIG. 7B, 7C).

### DETAILED DESCRIPTION OF THE INVENTION

Gene therapy holds great promise for improvements in the treatment of many diseases. However, this approach has been severely restricted by an inability to efficiently and selectively achieve transgene expression in appropriate target cells. Key limitations to the meaningful application of this new technology are the shortcomings of gene delivery agents (vectors) which have failed to show a capacity to specifically direct transgene expression to target cells. The importance of specific targeting has long been appreciated; in the last six years multiple reports have emerged describing a variety of targeting approaches, many of which are based on adenoviral (Ad) vectors, in view of their generally high in vivo gene delivery efficiency. Unfortunately, there is still a lack of evidence that a systemically administered vector can achieve truly specific and efficient transgene expression.

The present invention provides a system that improves the efficacy and specificity of achieving transgene expression in vivo using adenoviral vectors. By combining tropism modification to achieve transductional retargeting, and transcriptional control using a tissue-specific promoter, a highly synergistic improvement in target to non-target gene expression ratio was achieved.

The current invention dramatically improves the specificity of transgene expression, specifically in the context of gene delivery to the pulmonary vascular endothelium. The combination of transductional targeting to a pulmonary endothelial marker (angiotensin-converting enzyme, ACE) and an endothelial-specific promoter (for vascular endothelial growth factor receptor type 1, flt-1) resulted in a synergistic, 300,000-fold improvement in the selectivity of transgene expression for lung versus the usual site of vector sequestration, the liver. However, the basic concept of the present invention could be applied to gene delivery for many cell types. In this way, this approach could greatly enhance the utility of gene therapy strategies for virtually any disease process.

The combined targeting approach of the present invention could employ other target molecules and tissue-specific promoters in addition to the ones disclosed herein. For example, representative example of useful target molecules include receptors and other surface motifs known to be upregulated in tumors, e.g. epidermal growth factor (EGF), fibroblast growth factor (FGF), ErbB2 (Her-2), and Carcinoembryonic antigen (CEA). Similarly, receptors and surface accessible molecules present on various normal tissues could be exploited including PECAM E-selectin and ICAM on endothelial cells and the urokinase plasminogen activator receptor on airway epithelium. Furthermore, cytokine and other growth factor receptors known to be upregulated in various pathological states could also be exploited. In addition to known and recognized markers, recently discovered ligands (including peptides, single-chain antibodies and derivative thereof) identified by phage-panning technology or similar procedures could also be included—examples include the “SIGYPLP” peptide which has affinity for endothelium and the “SSS-10” peptides which has selectivity for airway epithelium.

The use of tissue specific promoters is an attractive means for controlling gene expression. Early efforts to exploit this technology in the context of adenoviral vectors were sometimes undermined when the promoter was placed in the adenoviral genome; ill defined cis or trans acting effects had the potential to interfere with promoter specificity (33). Recently, however, an increasing number of promoters that retain fidelity in the adenoviral genome are being described. Given the natural tropism of Ad for the liver and spleen, candidate tissue-specific promoters should have low activity in these organs.

Three candidate endothelial specific promoters have been evaluated—fit-1, ICAM-2 and von Willebrand factor (16). Of the three, fit-1 had an advantage in terms of both strength and specificity. Furthermore, recent studies have indicated that VEGF receptors are expressed in normal pulmonary endothelium where they play an important role the maintenance of pulmonary vascular integrity (34, 35). Thus the flt-1 promoter was a rational choice for the current study (and the promoter for VEGFR2/Flk-1 might similarly prove effective). However, as it is clearly shown in the present study, the use of this approach alone was limited by the low level of transduction of pulmonary endothelium by adenoviral vectors with native tropism. The full potential of this promoter was only realized in the context of tropism modification. In this regard, upregulation of both the expression of angiotensin converting enzyme and vascular endothelial growth factor receptors has been described in the vicinity of plexiform lesions associated with primary pulmonary hypertension (36, 37). Thus, the combined targeting approach presented in the current study may have particular relevance for the development of gene therapy for this disorder.

Many similar logical combinations of transductional and transcriptional approaches could be envisaged for other diseases, thus underlining the general importance of the paradigm established here.

The use of the flt-1 promoter in the current study has disease relevance in that both flt-1 and angiotensin converting enzyme are increased in the context of vascular remodelling in primary pulmonary hypertension. One of ordinary skill in the art would recognize that the double-targeting approach described herein should be applicable to other diseases as suitable ligands and promoters become known.

In addition, representative example of useful promoters include other endothelial-specific promoters such as promoters for preproendothelin, KDR; tumor specific promoters such as promoters for midkine, ErbB2, Muc1, Cox-2 and PSA; promoters for normal tissues such as promoters for K-18-airway epithelium and other CFIR expressing tissues; hepatocyte-specific promoter such a s promoter for albumen, and muscle-specific promoter such a s promoter for myosin.

As used herein, the term “transductional targeting” shall refer to the use of any strategy that alters the natural cell-binding and entry pathway of any viral or non-viral vector designed to delivery genes into cells.

As used herein, the term “transcriptional targeting” shall refer to any strategy that specifically uses any type of promoter in an effort to achieve cell-specific gene expression. The promoters include those that may be selectively induced by physiological stimuli (such as heat shock or hypoxia).

The instant invention is directed to an adenoviral vector that mediates increased gene delivery in vivo. This vector comprises: a targeting component that targets or directs the vector to specific target cells and a tissue-specific promoter that drives the expression of a transgene carried by the vector in the target cells. In general, the targeting component of the adenoviral vector can be a bi-specific molecule that binds to the knob protein or other capsid protein of the adenoviral vector and a molecule expressed on the target cells. Alternatively, the targeting component can be a targeting ligand incorporated into the fiber protein or other capsid protein of said adenoviral vector by genetic mutation.

One of ordinary skill in the art would readily recognize various methods of incorporating targeting ligand with specificity for target cellular markers into the major capsid proteins, fiber, penton or hexon protein of adenoviral vector. For example, short peptide ligands have been incorporated into either the carboxy terminal (41, 42) or the HI loop (43) of the knob domain of the adenoviral fiber protein. Minor capsid proteins such as pIIIa and pIX are also potential sites for targeting ligand incorporation. Moreover, U.S. Pat. No. 6,210,946 disclosed an adenovirus modified by replacing the adenovirus fiber protein with a fiber replacement protein comprising a) an amino-terminal portion comprising an adenoviral fiber tail domain; b) a chimeric fiber replacement protein; and c) a carboxy-terminal portion comprising a targeting ligand.

The present invention is also directed to an improved method of gene delivery by adenoviral vector, comprising the step of: contacting target cells with an adenoviral vector comprising a targeting component that targets the vector to specific target cells and a tissue-specific promoter that drives the expression of transgene carried by the vector in the target cells, wherein the adenoviral vector has enhanced targeting specificity to the target cells and results in reduced transgene expression in non-target cells. In general, the targeting component of the adenoviral vector can be a targeting ligand incorporated into the fiber protein or other capsid protein of said adenoviral vector by genetic mutation. Alternatively, the targeting component can be a bi-specific molecule that binds to the knob protein or other capsid protein of the adenoviral vector and a molecule expressed on the target cells. In one embodiment, when the target cells are pulmonary endothelial cells, the adenoviral vector comprises a vascular endothelial growth factor type 1 receptor promoter and a bi-specific antibody conjugate linking a Fab fragment of an anti-Ad5 knob antibody 1D6.14 with an antiangiotensin converting enzyme (ACE) antibody 9B9.

The following examples are given for the purpose of illustrating various embodiments of the invention and are not meant to limit the present invention in any fashion.

### EXAMPLE 1

Adenoviral Vector Construction

The luciferase reporter gene was obtained from the plasmid PGL3 basic (Promega), excised as a KpnI-SalI fragment (including the SV40 polyA signal) and ligated into the polylinker region of the adenoviral shuttle plasmid pShuttle, forming pShuttleLuc. The flt-1 promoter (−748 to +284) was excised from the plasmid pMV10-flt1 (16) using HindIII and XbaI, blunt ended then inserted into the HindIII site of pShuttleLuc, upstream of the luciferase gene, forming pShuttlefltLuc. A recombinant adenoviral genome was generated by homologous recombination with the pAdEasyl plasmid in E. coli as previously described (17). After confirmation of correct recombination the adenoviral genome w as lineraized using Pac1, then transfected into low passage 293 cells using Superfect (Qiagen Inc., Valencia Calif.) to generate the recombinant virus. Viral stocks were amplified in 293 cells and purified through two cesium chloride gradients using standard techniques (18). Plague titre and particle titer (based on OD 260) were determined by standard techniques. The control virus AdCMVLuc was constructed in a similar manner except the luciferase gene was inserted downstream of the CMV promoter in the plasmid pShuttleCMV (17). AdCMVCEA has been previously described (38). AdfltCEA was constructed by removing the luciferase gene from pShuttlefltLuc as an Xbal fragment, then ligating in the blunt ended CEA gene which was obtained from plasmid pGT37 (19) as a 2373 b p HindIII-NotI fragment.

### EXAMPLE 2

In Vitro Gene Transfer

The murine endothelial cell line 1P-1B was obtained from American Type Culture Collection (Manassas, Va.) and propagated in DMEM medium (Cellgro, Herndon, Va.) containing 10% fetal calf serum (FCS), penicillin and streptomycin. Cells were plated into 24 well plates at 50,000 cells per well. Twenty four hours later the cells were infected using virus diluted in DMEM containing 2% FCS for one hour, then infecting medium was removed and replaced with complete medium. Luciferase assay was performed 24 hours later using a Luciferase Assay System kit (Promega, Madison Wis.) according to the manufacturer's instructions, and a Femtomaster FB12 luminometer (Zylux Corporation, Maryville, Tenn.).

To evaluate gene transfer with AdfltCEA, cells were plated and infected as above. Forty eight hours later the cells were fixed using methanol/5% acetone, and stained using a rabbit anti CEA antibody (Chemicon, Temecula, Calif., Cat. #46912) followed by detection using biotinylated anti-rabbit anti-body, Vectastain ABC kit and diaminobenzidine (DAB) (Vector Laboratories) according to the manufacturer's instructions.

### EXAMPLE 3

Conjugate Construction And Characterization

Construction of Fab-9B9 and subsequent in vitro and in vivo validation has previously been described (12). Briefly, Fab and mAb 9B9 were derivatized with the bifunctional crosslinker N-succinimidyl 3-(2-pyridyldithio) propionate (SPDP; Pierce, Rockford, Ill.). SPDP was dissolved in 100% ethanol to a final concentration of 2 mg/ml, then combined with 9B9 or Fab in PBS at a molar ratio of 4 SPDP: 1 antibody and incubated with shaking at room temperature for 30 min. The pH of Fab was lowered by adding 0.1 volumes of 1 M sodium acetate, pH 4.5, then the Fab was reduced by adding 1 mg of solid dithiothreitol (DTT; Bio-Rad, Hercules, Calif.). After a 5 min incubation at room temperature the reduced Fab was passed through a PD10 column (Pharmacia, Uppsala, Sweden), equilibrated in borate buffer, then added immediately to the derivatized 9B9 and shaken at room temperature overnight. The conjugate mixture was subsequently purified by gel filtration on a HR 10/30 Superose 12 column (Pharmacia) in borate buffer pH 8.5. Monomeric Fab and 9B9 were discarded and fractions larger than 150 kDa were assessed for specificity.

### EXAMPLE 4

In Vivo Gene Transfer

For in vivo experiments, male Sprague-Dawley rats aged 6-8 weeks were obtained from Harlan Sprague Dawley Inc., Indianapolis, Ind. All experiments using animals were approved b y the University of Alabama at Birmingham Institutional Animal Care and Use Committee.

Luciferase gene delivery was carried out as follows. AdCMVLuc (5×10⁹ pfu) was complexed with 10 μg Fab-9B9 for 30 minutes at room temperature, then the total volume was brought to 200 μl with sterile normal saline. Rats were injected via the lateral tail vein, then sacrificed three days later. Organs (lungs, liver, spleen, kidney, heart) were harvested into 50 ml polypropylene tubes and snap frozen in ethanol/dry ice. For luciferase analysis, entire organs were ground to a fine powder using a mortar and pestle cooled in an ethanol/dry ice bath. One hundred milligrams of organ powder were weighed and placed in a 1.5 ml polypropylene tube. Subsequent processing for luciferase activity was performed using a Promega Luciferase Assay System kit (Promega, Madison Wis.). Tissue powders were lysed in 200 μl of cell lysis buffer and subjected to three freeze thaw cycles to ensure complete lysis. Tubes were centrifuged and supernatant analysed for luciferase activity according to the manufacturer's instructions. The protein concentration of lysate was determined using a Bio-Rad detergent compatible (DC) protein assay kit, according to the manufacturer's instructions.

For left ventricular injections, animals were anesthetized with ketamine, then the left ventricle localized using standard echocardiography. Vector was injected transcutaneously using a 25 gauge needle. Blood was withdrawn before and after dose administration to check needle-tip position, then repeat echo was performed.

Immunohistochemistry was carried out as follows. Rats were injected with 3×10¹⁰ pfu of either AdCMVCEA or AdfltCEA complexed to Fab-9B9. Three days later animals were sacrificed using CO₂. Lungs were perfused by inserting an 18G catheter into the right ventricle and making a small slit in the left ventricle. The pulmonary vascuature was perfused first with PBS/heparin (30 mls, 20 cm H₂O), then 30 mls neutral buffered formalin (10%, Formalin-Fresh, Fisher Scientific, Pittsburgh, Pa.). Lungs were then inflated by tracheal instillation of formalin, removed en-bloc and fixation continued overnight in formalin. Livers were removed and 1-2 mm strips were fixed in formalin overnight. These tissues were processed into paraffin the next day. The liver, lungs and spleen from any one animal were processed into a single block. Paraffin sections, cut at 4 μm, were heat mounted (58° C. for one hour) on glass slides (Fisherbrand Superfrost Plus). Slides were immunostained using an anti-CEA polyclonal rabbit antibody (Chemicon, Temecula, Calif., Cat. #46912) diluted 1:2000 with PBE buffer (1% BSA, 1 mM EDTA, 0.15 mM NaN₃, in PBS) for one hour at room temperature (RT), followed by detection with Alexa 488 (green fluorescence) goat anti-rabbit secondary antibody (Molecular Probes, Eugene, Oreg.) Secondary antibody incubations were also performed for one hour at room temperature. Nuclei were stained with Hoescht 33342 for 10 min at room temp. Immunofluorescent images were obtained using Olympus IX 70 inverted microscope with epifluorescence optics and Photometrics Sensys cooled CCD, high resolution, monochromatic camera (Roper Scientific; Tucson, Ariz.) and IPLab Spectrum Image Analysis software (Scanalytics; Fairfax, Va.).

Statistical comparisons between groups were made by logarithmic transformation of the data and Student's t-test

### EXAMPLE 5

Combined Transductional and Transcriptional Targeting

For the application of gene therapy to many common diseases, strategies to improve the fidelity of gene delivery are needed. To this end, the utility of combining transductional and transcriptional targeting approaches, in particular for gene delivery to pulmonary vascular endothelium, was assessed. A conjugate-based approach to target pulmonary endothelium in vivo via binding to angiotensin converting enzyme (ACE) was combined with the usage of flt-1 promoter that has a high degree of activity in, and specificity for, endothelial cells.

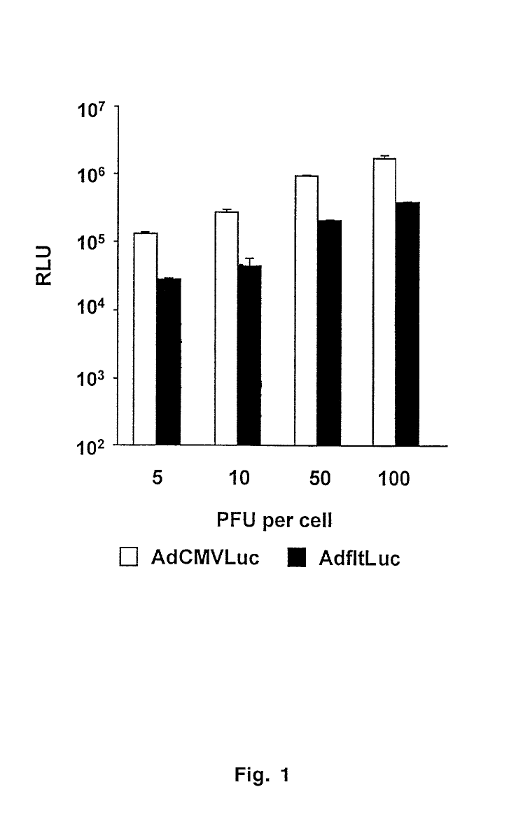

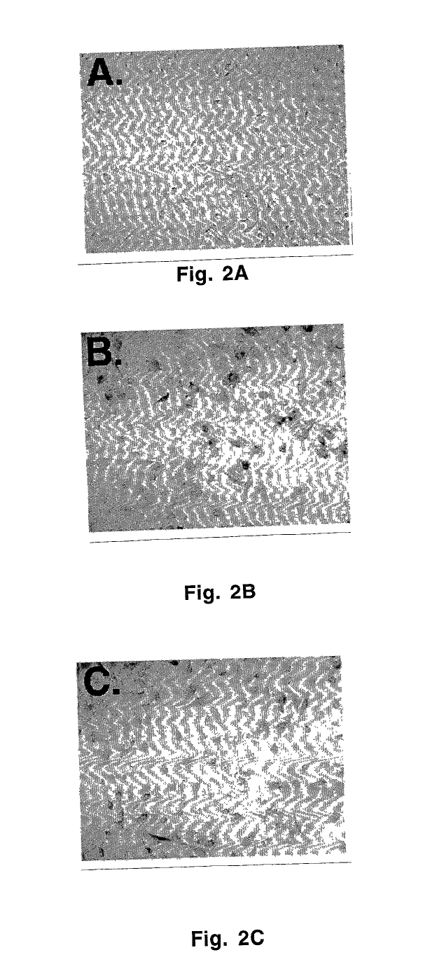

To enable sensitive detection of trangene expression in vivo an adenoviral vector containing the gene for firefly luciferase under the control of the flt-1 promoter (AdfltLuc) was constructed. Initially, this virus was compared with an adenoviral vector (AdCMVLuc) containing the same luciferase gene under the control of the strong, non-specific CMV promoter in infecting the 1P-1B murine endothelial cell line. Levels of luciferase activity obtained with AdfltLuc were approximately 20% of those obtained with AdCMVLuc (FIG. 1). A second adenoviral vector containing the gene for carcinoembryonic antigen (CEA) under the control of the flt-1 promoter (AdfltCEA) was also constructed because it was found that detection of carcinoembryonic antigen by immunohistochemistry was a very sensitive and specific method for localising transgene expression in vivo. This vector was evaluated alongside AdCMVCEA (containing carcinoembryonic antigen under the control of the CMV promoter) for its ability to transduce IP-IB cells. Immunohistochemical staining of cells infected with equal doses of vector showed comparable amounts of staining (FIG. 2). Thus the basic activity of the vectors in a relevant cellular substrate was confirmed.

To evaluate the double targeting concept, in vivo studies were used as the most relevant test system. A previously described transductional targeting approach using a bi-specific conjugate (Fab9B9) which was made by linking the Fab fragment of an anti-Ad5 knob antibody (1D6.14) (10) to the anti-angiotensin converting enzyme monoclonal antibody mAb 9B9 (20, 21) was used. To prepare targeting complexes, adenoviral vectors were incubated with Fab-9B9 for thirty minutes immediately prior to injection. Male Sprague-Dawley rats aged 8 weeks were used.

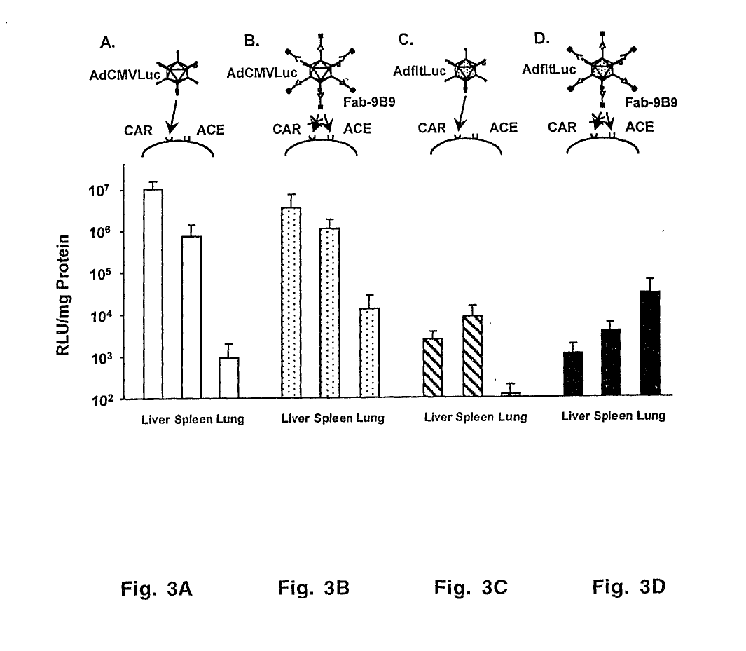

Initial studies were performed using the luciferase reporter system. Rats were injected by tail vein with either AdCMVLuc or AdfltLuc, each alone or in combination with Fab-9B9. Three days later rats were sacrificed, organs harvested and luciferase activity per mg protein was determined. Mean±SD of pooled raw data from two experiments is shown in FIG. 3, n=8−10 rats per group. Using the untargeted AdCMVLuc vector, transgene expression was seen mainly in the liver and spleen, as previously reported, with relatively little activity in the lungs. Addition of Fab-9B9 for transductional targeting to angiotensin converting enzyme expressed on pulmonary endothelium achieved a 15-fold increase in pulmonary transgene expression (p<0.001), and a 67% reduction in liver expression (p=0.028).

Substitution of AdfltLuc for AdCMVLuc, without Fab-9B9 resulted in a reduction in transgene expression in all organs. Importantly, when AdfltLuc was combined with Fab-9B9, the levels of transgene expression in the lungs were restored to levels achieved with the AdCMVLuc+Fab-9B9 combination (p<0.001, AdfltLuc vs AdfltLuc+Fab-9B9), and 30-fold higher than the levels achieved with AdCMVLuc alone. In contrast, adding Fab-9B9 to AdfltLuc reduced liver transgene expression (p=0.026, AdfltLuc vs. AdfltLuc+Fab-9B9), leading to a net 10,000-fold reduction compared with the use of AdCMVLuc alone. The double-targeting approach resulted in 27-fold higher gene expression in the lung than in the liver (relative light units (RLU)/mg protein, p<0.001) and 8-fold higher expression in the lung than in the spleen (p=0.003). The initial lung:liver ratio using the untargeted vector was 9×10⁻⁵; thus the double-targeting approach achieved an improvement in relative selectivity for the lung of over 300,000-fold. The lung:spleen ratio improved by >6,000-fold. Therefore, the combined transductional-transcriptional strategy had a strong synergistic effect that greatly improved the gene delivery profile compared with the use of either strategy alone.

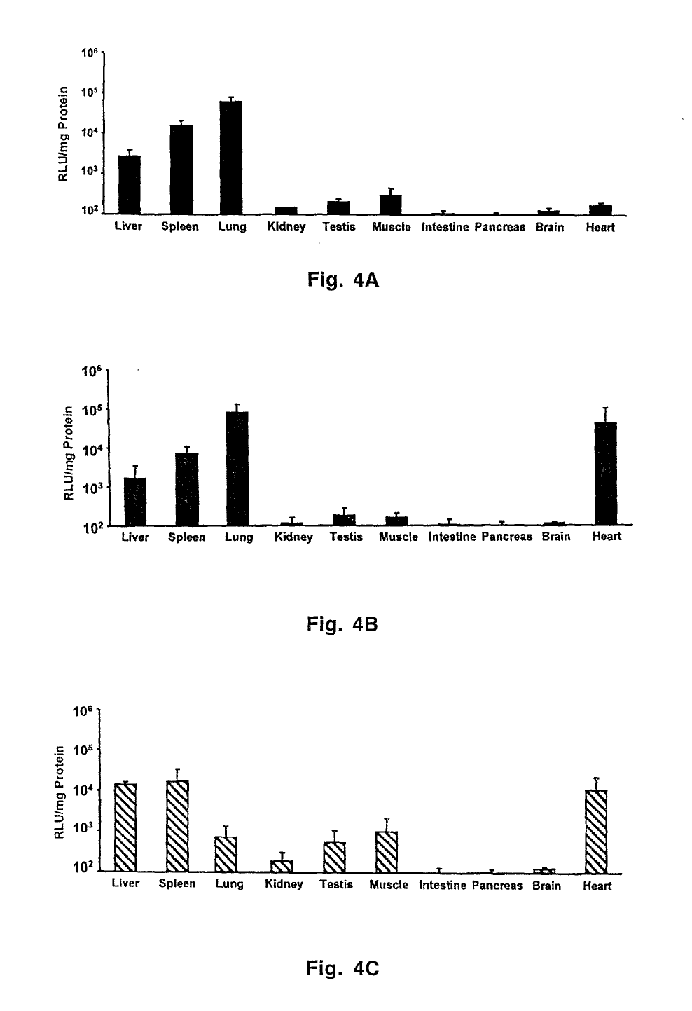

Next, transgene expression following either a tail vein or left ventricular (LV) injection of AdfltLuc/Fab-9B9 was compared. In this way, it was sought to determine whether targeting was influenced by the site of injection: the vector arrives at the pulmonary capillary bed soon after tail vein injection and much later after left ventricular administration. It was found that the distribution of transgene expression by the two approaches was very similar, with the exception that expression in the heart was higher with the left ventricular approach (FIG. 4). Thus, targeting of the vector disclosed herein did not depend on a first-pass effect. In principle, these findings have encouraging implications for the development of targeted adenoviral strategies for gene delivery to vascular beds other than the lung, provided suitably specific ligands can be identified.

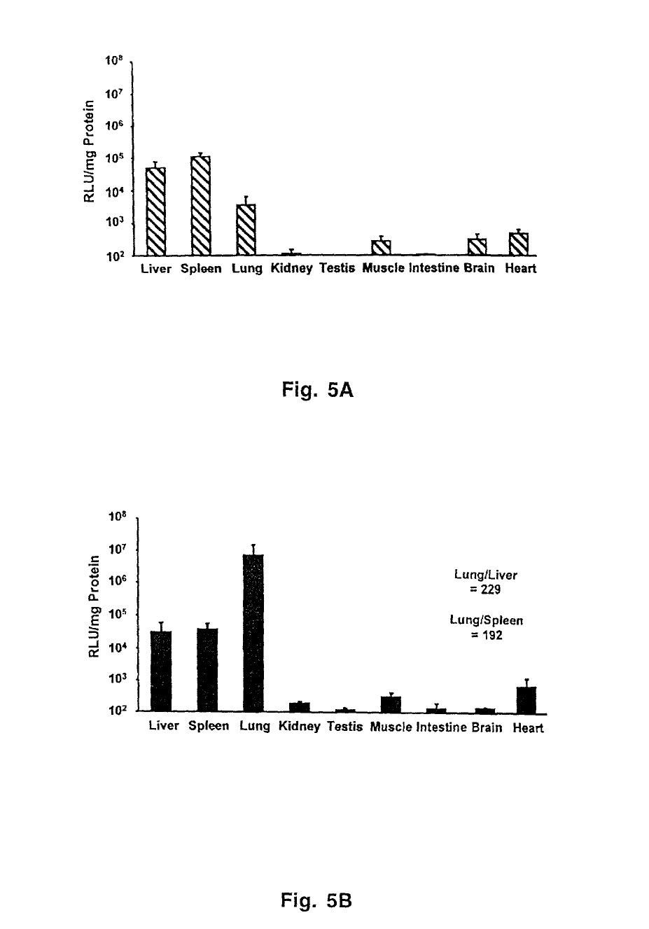

Recently, a threshold effect has been reported when adenoviral vectors are administered systemically (39, 40). This phenomenon arises because Kupffer cells which line the hepatic sinusoids phagocytose a large proportion (up to 90%) of vector at low doses, but become saturated at high doses, thereby allowing a greater fraction of the vector load to reach and transduce hepatocytes. A higher vector dose in the system disclosed herein was evaluated by injecting 3×10¹¹ viral particles (compared with 5×10¹⁰particles used in FIG. 3 and 1×10¹¹ in FIG. 4). Again, a significant improvement in pulmonary targeting was noted (FIG. 5). An even greater improvement of ˜200 in lung:liver and lung:spleen ratios was found at this dose. This may reflect a threshold effect whereby the higher dose yielded a proportionately greater expression in the target site because of Kupffer cell saturation.

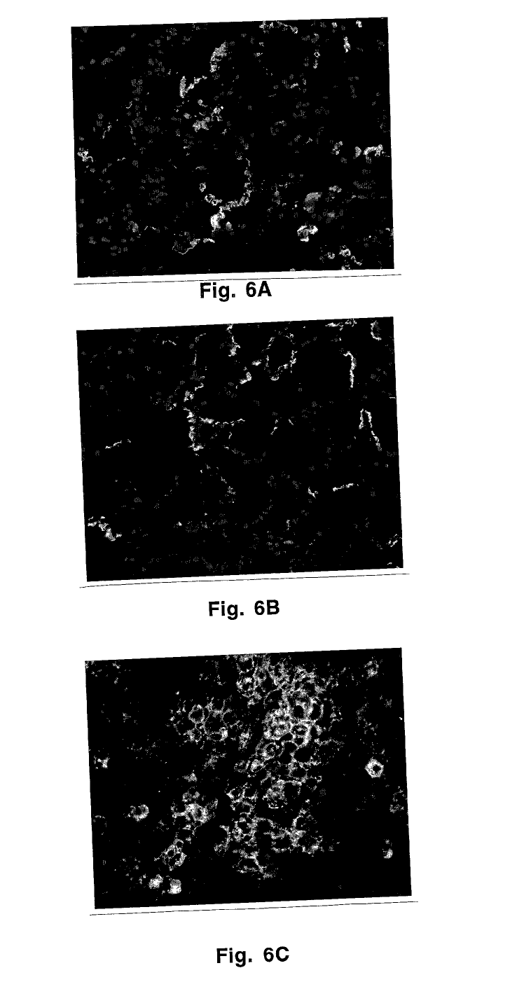

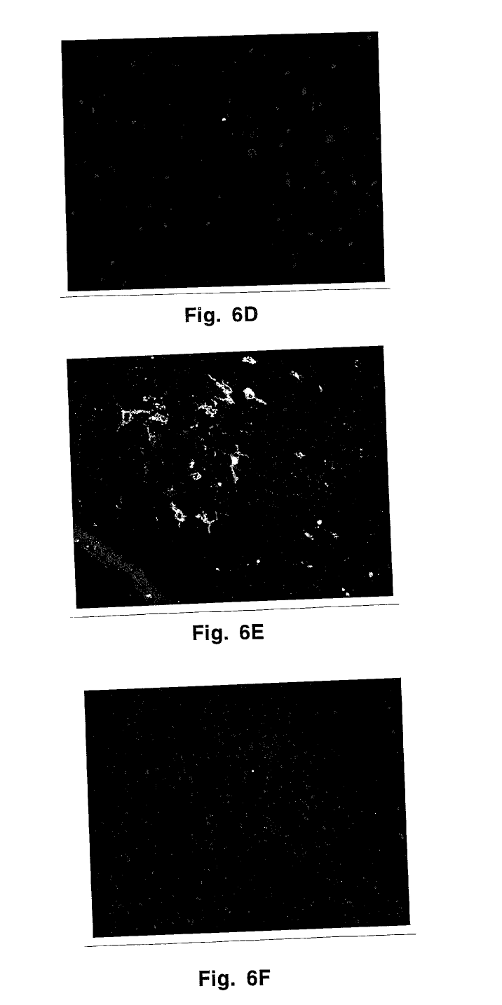

To achieve further confirmation of the efficacy of the double targeting strategy, and to assess the distribution of transgene expression within the organs, delivery of the carcinoembryonic antigen gene was examined with immunohistochemistry. ACEtargeted AdCMVCEA or AdfltCEA (3×10¹⁰ pfu) was administered by tail vein injection into rats, then the animals were sacrificed three days later. Lungs were perfused and fixed in inflation for 24 hours using 10% buffered formalin, livers and spleens were cut into 2 mm strips and similarly fixed. Paraffin sections were stained with a rabbit anti-carcinoembryonic antigen antibody and signal detected using Alexa 488-tagged goat anti-rabbit antibody (green fluorescence) and nuclei were stained using Hoescht 33342 (blue fluorescence) as shown in FIG. 6.

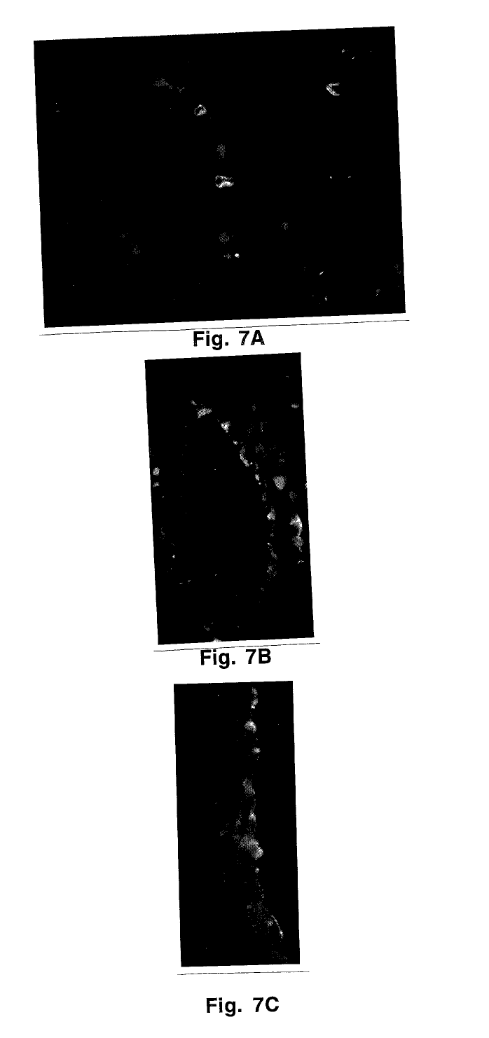

In rats that received the AdCMVCEA/Fab-9B9 combination, positive signal was readily detected in small pulmonary vessels, alveolar capillaries and hepatocytes as previously reported (FIG. 6A, C). Signal was also readily detected in the spleen (FIG. 6E). For rats that received the AdfltCEA/Fab-9B9 combination, signal was again readily detected in alveolar capillaries, to a degree at least comparable to or slightly more widespread than that seen with the AdCMVCEA/Fab-9B9 combination (FIG. 6B). In these animals, transgene expression was seen in at least 50% of alveolar walls. However, no signal was seen in the livers or spleens of these animals (FIG. 6D, F). No signal was seen in the negative controls, consisting of sections incubated with no primary antibody, and sections from an uninfected rat stained with anti-carcinoembryonic antigen antibody (data not shown). High power views clearly show staining within capillary loops in alveolar walls and in the endothelial layer of small vessels (FIG. 7). No signal was seen in organs from rats that received AdfltCEA alone. In rats that received AdCMVCEA alone, signal was seen in liver and spleen but not lung, as previously reported. Signal was also seen in alveolar capillaries after LV injection of ACE-targeted vector (data not shown). Thus, these studies confirmed the findings of the luciferase experiments: that the double targeting strategy achieved a substantial synergistic improvement in the specificity of transgene expression for the target site.

In addition to assessing transgene expression, haematoxylin and eosin (H & E) stained sections of the rat tissues were also examined to evaluate inflammatory responses. The sections of lung tissue from the rats that received either AdCMVCEA/Fab-9B9 or AdfltCEA/Fab-9B9 did not show any significant inflammatory changes compared to sections obtained from a control, uninfected rat. Sections of liver tissue from the rats that received either vector complex had multiple subtle histopathologic changes such as increased numbers of mitotic figures in hepatocytes, scattered hepatocytes with cytoplasmic vacuoles, scattered individual apoptotic or necrotic hepatocytes and prominent Kupffer cells (data not shown). The spleens had evidence of increased extramedullary hematopoiesis. These changes are consistent with previous findings in this model, and importantly showed no significant inflammatory response in the pulmonary target site. The hepatic changes are probably due to an early innate response to vector particles and an early response to low levels of viral gene expression.

In summary, the successful in vivo combination of transductional and transcriptional targeting approaches reported herein improves the prospects for gene therapy for pulmonary vascular disease and provides an important proof-of principle for further vector development generally. The ACE-targeting/flt-1 promoter approach has the potential to improve pulmonary vascular gene therapy while reducing the potential for transgene-induced toxicity.

To date, various conjugate-based transductional adenoviral-targeting strategies have been reported, including several which improve gene delivery to endothelial cells, by targeting to FGF receptors (22), integrins (23), E-selectin (24) or through the use of a novel ligand identified by bacteriophage panning (25). However, none of these approaches has shown specific transduction of endothelium in vivo. Using a strategy to target systemically administered adenoviral to FGF receptors, Gu et al achieved a reduction in liver transgene expression and an associated reduction in hepatic toxicity, but specific retargeting was not confirmed (26). Using the FGF approach to deliver a suicide gene in a loco-regional intraperitoneal murine model of ovarian carcinoma, Rancourt et al showed enhanced therapeutic outcome, but again, specificity of targeting was not assessed (27).

An alternate transductional targeting approach is the direct genetic mutation of the adenoviral knob domain to incorporate specific targeting ligands (11). This approach is attractive because it potentially avoids the complexity of the “two-component” conjugate system. However, results to date have been limited to the expansion of tropism via the incorporation of non-specific ligands such as RGD (30) or polylysine (31). Simultaneous ablation of native tropism with true retargeting has not been reported. Structural constraints limit the size of ligands that can be genetically incorporated into the knob, but newer approaches such as fiber replacement strategies may overcome this restriction (32). Nevertheless, evidence is emerging that ablation of CAR recognition alone will be insufficient to substantially reduce hepatic transgene expression, either because of residual penton RGD-integrin interactions or other non-specific cell entry mechanisms. Thus, even in the context of these technological improvements, some additional measures of control are required.

The angiotensin converting enzyme-targeting approach disclosed herein is the only technique described that has a degree of fidelity upon systemic administration. The specificity of the approach is achieved due to 1) the large size of the pulmonary vascular bed, 2) the fact that all pulmonary capillary endothelial cells express angiotensin converting enzyme (29), and 3) the accessibility of pulmonary angiotensin converting enzyme from the circulation. Moreover, angiotensin converting enzyme-targeting does not depend on a first-pass effect. Thus, although angiotensin converting enzyme is expressed elsewhere in less accessible areas such as the proximal tubular epithelium of the kidney, it has been shown to be an ideal target for pulmonary drug or gene delivery. In addition, levels of circulating angiotensin converting enzyme are at least 100-fold less than in the rat lung, and angiotensin converting enzyme is not expressed on the endothelium of hepatic sinusoids. However, when used alone, significant hepatocyte transgene expression still occurred, thus necessitating a combined approach of transduction and transcription control.

The transductional-transcriptional approach described herein could easily be combined with other technological advances such as genetic capsid modifications, fully deleted (“gutless”) vectors, and approaches to avoid sequestration of the vector by the reticuloendothelial system. Such combinations will further optimize the specificity and efficacy of gene delivery.

The following references were cited herein:

1. Moraes and Loscalzo. 1997. Clin Cardiol 20(8):676-82.

2. Russell. 2000. J Gen Virol 81(Pt 11):2573-2604.

3. Rodman et al. 1997. American Journal of Respiratory Cell & Molecular Biology 16(6):640-9.

4. Huard et al. 1995. Gene Therapy 2(2):107-15.

5. Bergelson et al. 1997. Science 275(5304):1320-3.

6. Tomko et al. 1997. Proc. Natl. Acad. Sci. USA 94(7):3352-6.

7. Yee et al. 1996. Human Gene Therapy 7(10):1251-7.

8. Wickham. 2000. Gene Ther 7(2):110-4.

9. Wickham et al. 1993. Cell 73(2):309-19.

10. Douglas et al. 1996. Nature Biotechnology 14:1574-1578.

11. Krasnykh et al. 2000. Mol Ther 1(5 Pt 1):391-405.

12. Reynolds et al. 2000. Mol Ther 2(6):562-578.

13. Nettelbeck et al. 2000. Trends Genet 16(4):174-81.

14. Adachi et al. 2000. Cancer Research In Press.

15. Koeneman et al. 2000. World J Urol 18(2):102-10.

16. Nicklin et al. 2001. Hypertension Submitted.

17. He et al. 1998. Proc Natl Acad Sci USA 95(5):2509-14.

18. Graham and Prevec. 1991. Manipulation of adenovirus vectors. In Murray et al. editors. Methods in Molecular Biology. Humana Press, Clifton, N.J. 109-129.

19. Conry et al. 1994. Cancer Res 54(5):1164-8.

20. Danilov et al. 1991. Lab Invest 64(1):118-24.

21. Atochina et al. 1998. Am J Physiol 275(4 Pt 1):L806-17.

22. Reynolds et al. 1998. Tumor Targeting 3:156-168.

23. Wickham et al. 1996. J Virol 70(10):6831-8.

24. Harari et al. 1999. Gene Ther 6(5):801-7.

25. Nicklin et al. 2000. Circulation 102(2):231-7.

26. Gu et al. 1999. Cancer Res 59(11):2608-14.

27. Rancourt et al. 1998. Clin Cancer Res 4(10):2455-2461.

28. Schneider et al. 2000. Gene Ther 7(18):1584-92.

29. Franke et al. 1997. CD143 Workshop: Angiotensin-I-converting enzyme (CD143) on endothelial cells in normal and in pathological conditions. In Kishimoto, et al., editors. Leukocyte Typing VI. Garland Publishing Inc., New York. 749-751.

30. Dmitriev et al. 1998. Journal of Virology 72(12):9706-9713.

31. Wickham et al. 1997. Journal of Virology 71(11):8221-58229.

32. Krasnykh, V. 2001. Fiber repalcement.

33. Ring et al. 1996. Gene Ther 3(12):1094-103.

34. Partovian et al. 2000. Am J Respir Cell Mol Biol 23(6):762-71.

35. Kasahara et al. 2000. J Clin Invest 106(11):1311-9.

36. Schuster et al. 1996., Am J Respir Crit Care Med 154 (4 Pt 1):1087-91.

37. Hirose et al. 2000. Pathol Int 50(6):472-9.

38. Raben et al. 1996. Gene Ther. 3:567-580.

39. Bristol et al. 2000. Mol. Ther. 2:223-232.

40. Tao et al. 2001. Mol. Ther. 3:28-35.

41. Wickham et al. 1996. Nat Biotechnol 14:1570-3.

42. Wickham et al. 1997. Journal of Virology 71:8221-8229.

43. Dmitriev et al. 1998. J Virol 72:9706-13.

Any patents or publications mentioned in this specification are indicative of the levels of those skilled in the art to which the invention pertains. Further, these patents and publications are incorporated by reference herein to the same extent as if each individual publication was specifically and individually indicated to be incorporated by reference.

One skilled in the art will appreciate readily that the present invention is well adapted to carry out the objects and obtain the ends and advantages mentioned, as well as those objects, ends and advantages inherent herein. The present examples, along with the methods, procedures, treatments, molecules, and specific compounds described herein are presently representative of preferred embodiments, are exemplary, and are not intended as limitations on the scope of the invention. Changes therein and other uses will occur to those skilled in the art which are encompassed within the spirit of the invention as defined by the scope of the claims.

What is claimed is:

1. An adenoviral vector that mediates increased gene delivery in vivo comprising:
a targeting component that targets said vector to specific target cells; and
a tissue-specific promoter that drives the expression of a transgene carried by said vector in said target cells.

2. The adenoviral vector of claim 1, wherein said targeting component is selected from the group consisting of a targeting ligand incorporated into the fiber protein of said adenoviral vector by genetic mutation, a targeting ligand incorporated into a capsid protein of said adenoviral vector by genetic mutation, and a bi-specific molecule that binds to the knob protein of said adenoviral vector and a molecule expressed on said target cells.

3. The adenoviral vector of claim 2, wherein said bi-specific molecule is a bi-specific antibody conjugate linking a Fab fragment of an anti-Ad5 knob antibody with an anti-angiotensin converting enzyme antibody.

4. The adenoviral vector of claim 3, wherein said anti-Ad5 knob antibody is 1D6.14 and said anti-angiotensin converting enzyme antibody is 9B9.

5. The adenoviral vector of claim 4, wherein said tissue-specific promoter is selected from the group consisting of vascular endothelial growth factor type 1 receptor promoter, ICAM-2 promoter, vonwillebrand factor promoter and vascular endothelial growth factor receptor promoter.

6. The adenoviral vector of claim 5, wherein said target cells are pulmonary endothelial cells.

7. A method of gene delivery by adenoviral vector, comprising the step of:
contacting target cells with an adenoviral vector comprising a targeting component that targets said vector to specific target cells and a tissue-specific promoter that drives the expression of a transgene carried by said vector in said target cells, wherein said adenoviral vector has increased targeting specificity to said target cells and results in reduced transgene expression in nontarget cells.

8. The method of claim 7, wherein the targeting component of said adenoviral vector is selected from the group consisting of a targeting ligand incorporated into the fiber protein of said adenoviral vector by genetic mutation, a targeting ligand incorporated into a capsid protein of said adenoviral vector by genetic mutation, and a bi-specific molecule that binds to the knob protein of said adenoviral vector and a molecule expressed on said target cells.

9. The method of claim 8, wherein said bi-specific molecule is a bi-specific antibody conjugate linking a Fab fragment of an anti-Ad5 knob antibody with an anti-angiotensin converting enzyme antibody.

10. The method of claim 9, wherein said anti-Ad5 knob antibody is 1D6.14 and said anti-angiotensin converting enzyme antibody is 9B9.

11. The method of claim 10, wherein the tissue-specific promoter of said adenoviral vector is selected from the group consisting of vascular endothelial growth factor type 1 receptor promoter, ICAM-2 promoter, vonWillebrand factor promoter and vascular endothelial growth factor receptor promoter.

12. The method of claim 11, wherein the target cells are pulmonary endothelial cells.
